(19) United States
(12) Patent Application Publication
(10) Pub. No.: US 2026/0305998 A1
(43) Pub. Date: Oct. 8, 2026

(54) TRAINING SUPPORT INFORMATION PROVIDING DEVICE

(71) Applicants: Aisin Corporation, Kariya (JP); Tokai University Educational System, Tokyo (JP)

(72) Inventors: Hideaki YAMAGUCHI, Kariya-shi (JP); Eiji FUJIOKA, Kariya-shi (JP); Yoshika SEKINE, Hiratsuka-shi (JP)

(73) Assignees: Aisin Corporation, Kariya (JP); Tokai University Educational System, Tokyo (JP)

(21) Appl. No.: 19/573,522

(22) Filed: Mar. 20, 2026

(30) Foreign Application Priority Data
Apr. 3, 2025 (JP) 2025-062070

(51) Int. Cl. A61B 5/16; A61B 5/00; A61B 5/02; A63B 24/00; G01N 33/497

(52) U.S. Cl. CPC A61B 5/165; A61B 5/02007; A61B 5/443; A61B 5/4866; A61B 5/7264; A63B 24/0075; A61B 2505/09; G01N 33/497

(57) ABSTRACT

A training support information providing device according to the embodiment includes: an acquisition unit that acquires emission amount information related to an emission amount of a plurality of types of skin gases emitted from a skin of a subject; a calculation unit that calculates at least one objective index value indicating a ratio of an emission amount of specific skin gas selected from the plurality of types of skin gases based on the emission amount information; a determination unit that determines a stress state of the subject based on the at least one objective index value and determines training suitability for the subject based on the determined stress state; a generation unit that generates training support information related to the subject based on the training suitability for the subject; and an output unit that outputs the training support information.

### CROSS REFERENCE TO RELATED APPLICATIONS

This application is based on and claims priority under 35 U.S.C. § 119 to Japanese Patent Application No. 2025-062070, filed on Apr. 3, 2025, the entire content of which is incorporated herein by reference.

### TECHNICAL FIELD

This disclosure generally relates to a training support information providing device.

### BACKGROUND DISCUSSION

In general, in training, an athlete drives himself/herself into a stress state by repeatedly applying a load to the mind and body, and enhances their mental and physical functions in a recovery process from the stress state, which leads to improvement in exercise performance. Therefore, in order to improve the function to the maximum extent, some athletes perform training with a load at a limit level just before leading to injury or physical breakdown.

In recent years, various technologies have been proposed in order to support such training of the athlete. For example, there is a technology for determining an injury risk level based on a magnitude of divergence between a subjective evaluation and an objective evaluation for a fatigue level of each muscle and supporting pre-adjustment of training intensity, and there is a technology for simultaneously measuring and managing exercise information such as a health state determined based on heart rate, blood pressure, and amount of perspiration during exercise, an exercise load amount, and a form, which are considered effective for training support (See JP 2021-104304 A and JP 2008 003964 A).

However, in the related art, for example, in a case where the evaluation is complicated in sports involving complex movements, advanced data analysis skills and knowledge may be required for practical use in training.

A need thus exists for a training support information providing device which is not susceptible to the drawback mentioned above.

### SUMMARY

A training support information providing device includes: an acquisition unit that acquires emission amount information related to an emission amount of a plurality of types of skin gases emitted from a skin of a subject; a calculation unit that calculates at least one objective index value indicating a ratio of an emission amount of specific skin gas selected from the plurality of types of skin gases based on the emission amount information; a determination unit that determines a stress state of the subject based on the at least one objective index value and determines training suitability for the subject based on the determined stress state; a generation unit that generates training support information related to the subject based on the training suitability determination for the subject; and an output unit that outputs the training support information.

### BRIEF DESCRIPTION OF THE DRAWINGS

The foregoing and additional features and characteristics of this disclosure will become more apparent from the following detailed description considered with the reference to the accompanying drawings, wherein:

FIG. 1 is a diagram illustrating an example of a hardware configuration of a training support information providing device according to an embodiment;

FIG. 2 is a diagram illustrating an example of a detection position of skin gas according to the embodiment;

FIG. 3 is a diagram illustrating an example of a functional configuration of a training support information providing device according to the embodiment;

FIG. 4 is a diagram illustrating an example of a mental and physical state two-dimensional map showing a relationship among a vascular index value, a metabolic index value, and a stress state;

FIG. 5 is a diagram illustrating another example of a mental and physical state two-dimensional map showing a relationship among a vascular index value, a metabolic index value, and a stress state;

FIG. 6 is a diagram illustrating still another example of a mental and physical state two-dimensional map showing a relationship among a vascular index value, a metabolic index value, and a stress state;

FIG. 7 is an example of a two-dimensional map showing a relationship between a training intensity of a subject and a time-series transition of a stress state, and is a diagram illustrating an example of training suitability classification information;

FIG. 8 is another example of a two-dimensional map showing a relationship between a training intensity of a subject and a time-series transition of a stress state, and is a diagram illustrating another example of training suitability classification information;

FIG. 9 is still another example of a two-dimensional map showing a relationship between a training intensity of a subject and a time-series transition of a stress state, and is a diagram illustrating still another example of training suitability classification information;

FIG. 10 is still another example of a two-dimensional map showing a relationship between a training intensity of a subject and a time-series transition of a stress state, and is a diagram illustrating still another example of training suitability classification information;

FIG. 11 is a flowchart illustrating an example of a flow of training support information generation processing;

FIG. 12 is a diagram illustrating an example of a form of providing training support information to a subject;

FIG. 13 is a diagram illustrating an example of training suitability classification information according to a second embodiment;

FIG. 14 is a diagram illustrating an example of a relationship between a vascular index value and a stress value; and

FIG. 15 is a diagram illustrating an example of a relationship between a metabolic index value and a physical stress value.

### DETAILED DESCRIPTION

Hereinafter, a training support information providing system and a training support information providing device according to the present embodiment will be described with reference to the drawings. The configurations of the embodiments to be described below and the functions and effects provided by the configurations are merely examples, and the present disclosure is not limited to the following description.

### First Embodiment

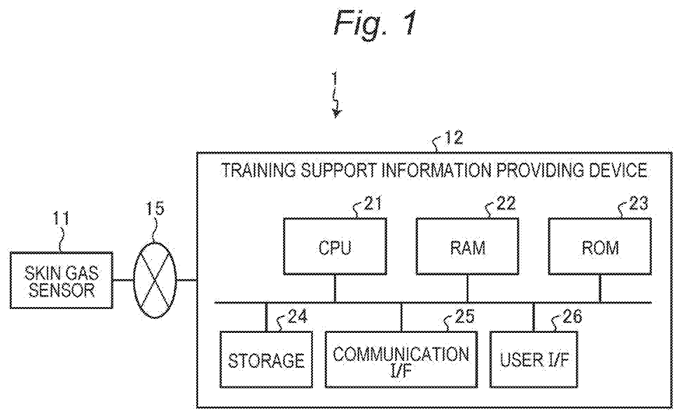

FIG. 1 is a diagram illustrating an example of a hardware configuration of a training support information providing system 1 according to a first embodiment. The training support information providing system 1 according to the present embodiment includes a skin gas sensor 11 and a training support information providing device 12.

The skin gas sensor 11 is a sensor that detects skin gas emitted from a skin of a subject. The skin gas sensor 11 can detect skin gas generated in association with activities for maintaining homeostasis in a human body, and can detect at least a vascular-related gas related to vasomotor activity (constriction or dilation of blood vessels), and a metabolic-related gas related to metabolic activity.

The vascular-related gas may be, for example, hexanoic acid, propionic acid, or the like.

The metabolic-related gas may be, for example, acetone, acetic acid, acetaldehyde, or the like.

The specific configuration of the skin gas sensor 11 is not particularly limited, but for example, an oxide semiconductor sensor, a quartz crystal microbalance (QCM) sensor, a field effect transistor (FET) sensor, a membrane-type surface stress sensor, or the like may be used as the skin gas sensor 11. Furthermore, skin gas emission amount data obtained by quantitatively measuring skin gas collected by a passive flux sampler (PFS) for each skin gas type using a gas chromatography-mass spectrometry (GC-MS) device or the like also be used as data of the skin gas sensor 11. The PFS is a small device that collects skin gas diffusing from a skin surface of a subject by causing the skin gas to be adsorbed onto a solid collecting material, in which the collected skin gas is then thermally or chemically desorbed from the collecting material to be subjected to chemical analysis, for example, a skin gas sampler (MonoTrap (registered trademark) SG DCC18) manufactured by GL Sciences Inc. may be used as the PFS.

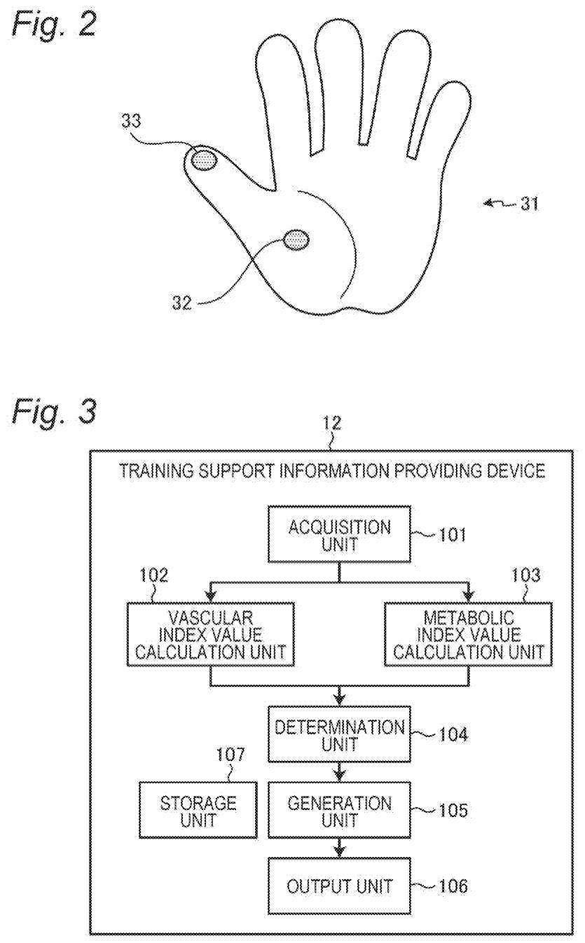

FIG. 2 is a diagram illustrating an example of a detection position of skin gas according to the embodiment. The position where the skin gas is detected by the skin gas sensor 11 according to the present embodiment is a palm 31 of the subject, and in particular, is preferably a thenar eminence portion 32 or a fingertip portion 33. Since these regions have many capillaries, are usually hairless, and have little subcutaneous fat, the emission amount of skin gas is large, and the skin gas can be detected relatively stably. In FIG. 2, a fingertip of a thumb is illustrated as the fingertip portion 33, and the fingertip portion 33 may be a fingertip of another finger (an index finger, a middle finger, a ring finger, or a little finger).

The detection position, specific configuration, installation position, and the like of the skin gas sensor 11 are not limited to the above. For example, the detection position may be the back of the knee or the like.

The training support information providing device 12 is connected to the skin gas sensor 11 via a predetermined network 15, and executes various types of information processing for detecting stress of the subject based on a detection result of the skin gas sensor 11.

The training support information providing device 12 exemplified here is configured using a general-purpose information processing device (a computer), and includes a central processing unit (CPU) 21, a random access memory (RAM) 22, a read only memory (ROM) 23, a storage 24, a communication interface (I/F) 25, and a user I/F 26.

The CPU 21 executes predetermined arithmetic processing using the RAM 22 as a work area in accordance with a program stored in the ROM 23 or the storage 24. The storage 24 is a non-volatile memory such as a solid state drive (SSD) or a hard disk drive (HDD), and enables writing and reading of programs, data necessary for processing by the CPU 21, data generated by processing of the CPU 21, and the like. The communication I/F 25 is a device that enables communication with other electronic devices such as the skin gas sensor 11 connected via a predetermined network 15. The user I/F 26 is a device that enables reception of input from a user (a user of the training support information providing system 1 or the like), output of information to the user, and the like, and may be, for example, a keyboard, a pointing device, a touch panel mechanism, a display, a speaker, a microphone, or the like. The CPU 21, the RAM 22, the ROM 23, the storage 24, the communication I/F 25, and the user I/F 26 are connected via a data bus.

The hardware configuration of the training support information providing device 12 is not limited to the above, and may be configured using, for example, a field programmable gate array (FPGA), an application specific integrated circuit (ASIC), or the like, or may be a system in which a plurality of computers operates in cooperation.

FIG. 3 is a diagram illustrating an example of a functional configuration of the training support information providing device 12 according to the embodiment. The training support information providing device 12 according to the present embodiment includes an acquisition unit 101, a vascular index value calculation unit 102, a metabolic index value calculation unit 103, a determination unit 104, a generation unit 105, an output unit 106, and a storage unit 107. These functional units 101 to 107 are configured by cooperation of hardware elements and software elements (programs, firmware, and the like) constituting the training support information providing device 12. Furthermore, at least a part of these functional units 101 to 107 may be configured by dedicated hardware (circuit or the like).

The acquisition unit 101 acquires, from the skin gas sensor 11, emission amount information regarding the emission amount of skin gas emitted from the skin of the subject. The acquisition unit 101 according to the present embodiment acquires the vascular-related gas information indicating the emission amount of each of a plurality of types of vascular-related gases and the metabolic-related gas information indicating the emission amount of each of a plurality of types of metabolic-related gases. The emission amount may be, for example, a value indicating the amount of gas (detected by the skin gas sensor 11) emitted per predetermined unit time.

The vascular-related gas information includes, for example, an emission amount of hexanoic acid, and an emission amount of propionic acid.

The metabolic-related gas information includes, for example, an emission amount of acetone, an emission amount of acetic acid, and an emission amount of acetaldehyde.

The emission amount information acquired by the acquisition unit 101 may include information regarding an emission amount of skin gas other than the vascular-related gas and the metabolic-related gas, for example, other skin gas or the like emitted in accordance with homeostasis maintenance activities in the body.

The vascular index value calculation unit 102 calculates vascular index value based on the vascular-related gas information acquired by the acquisition unit 101. The vascular index value is a value indicating a ratio of an emission amount of a specific vascular-related gas selected from a plurality of types of vascular-related gases to a total emission amount of the plurality of types of vascular-related gases. For example, when a plurality of types of vascular-related gases are hexanoic acid and propionic acid, a specific vascular-related gas is hexanoic acid, an emission amount of hexanoic acid is set to A1, and an emission amount of propionic acid is set to A2, a vascular index value BV can be calculated by the following expression (1).

$$BV = A 1 / ( A 1 + A 2 ) \qquad ( 1 )$$

That is, the vascular index value BV may be a ratio of an emission amount of hexanoic acid to a total emission amount of hexanoic acid and propionic acid. A method for calculating the vascular index value BV is an example, and the selection of a plurality of types of vascular-related gases and the selection of a specific vascular-related gas are not limited to the above. For example, the vascular index value BV may be a ratio of an emission amount of propionic acid to a total emission amount of hexanoic acid and propionic acid (BV′=A2/(A1+A2)).

As described above, by using the vascular index value BV, which is the ratio of the emission amount of the specific vascular-related gas to the total emission amount of the plurality of vascular-related gases, it is possible to suppress the influence due to variation in the emission amount of skin gas caused by climatic conditions or subject-specific factors (for example, physical constitution, mental state, and the like).

The metabolic index value calculation unit 103 calculates metabolic index value based on the metabolic-related gas information acquired by the acquisition unit 101. The metabolic index value is a value indicating a ratio of an emission amount of a specific metabolic-related gas selected from a plurality of types of metabolic-related gases to a total emission amount of the plurality of types of metabolic-related gases. For example, when a plurality of types of metabolic-related gases are acetone, acetic acid, and acetaldehyde, a specific metabolic-related gas is acetone, an emission amount of acetone is B1, an emission amount of acetic acid is B2, and an emission amount of acetaldehyde is B3, a metabolic index value ME can be calculated by the following expression (2).

$$M E = B 1 / ( B 1 + B 2 + B 3 ) \qquad ( 2 )$$

That is, the metabolic index value ME may be a ratio of an emission amount of acetone to a total emission amount of acetone, acetic acid, and acetaldehyde. A method for calculating the metabolic index value ME is an example, and the selection of a plurality of types of metabolic-related gases and the selection of a specific metabolic-related gas are not limited to the above. For example, the metabolic index value ME may be a ratio of an emission amount of a total emission amount of acetic acid and aldehyde to the total emission amount of acetone, acetic acid, and acetaldehyde (ME′=(B2+B3)/(B1+B2+B3)).

As described above, by using the metabolic index value ME, which is the ratio of the emission amount of the specific metabolic-related gas to the total emission amount of a plurality of metabolic-related gases, it is possible to suppress the influence due to variation in the emission amount of skin gas caused by climatic conditions or subject-specific factors (for example, physical constitution, mental state, and the like).

The determination unit 104 determines the stress state of the subject based on at least one index value. Specifically, based on the vascular index value BV calculated by the vascular index value calculation unit 102 and the metabolic index value ME calculated by the metabolic index value calculation unit 103, the determination unit 104 determines whether or not the subject is in a stress state of feeling a predetermined degree or more of stress.

The vascular index value BV and the metabolic index value ME, which are indices used for determination by the determination unit 104, are index values calculated based on chemical component analysis, and thus can be referred to as objective index values. Furthermore, the vascular index value BV and the metabolic index value ME are examples of objective index values. The determination unit 104 may determine whether or not the subject is in the stress state based on index values other than the vascular index value BV and the metabolic index value ME.

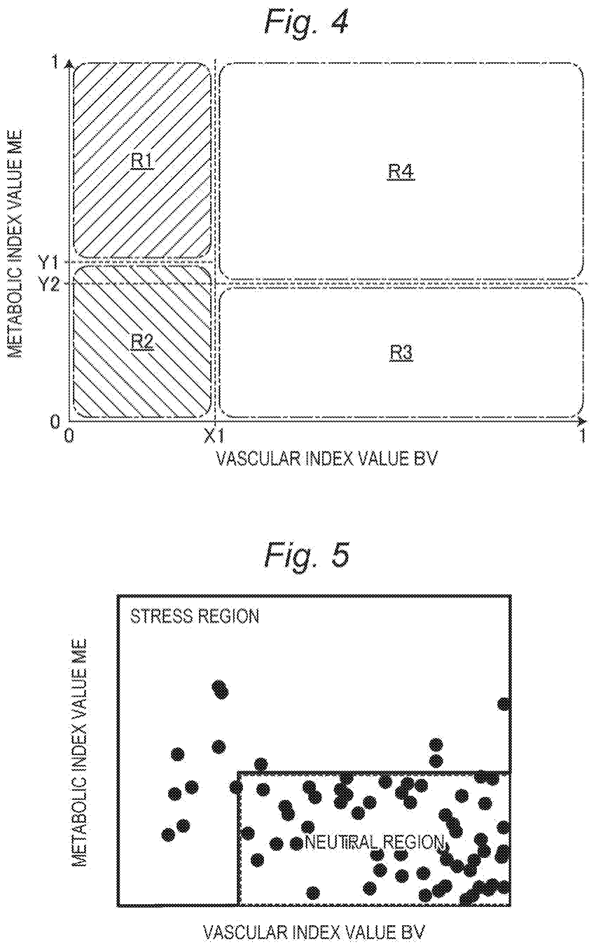

FIG. 4 is a diagram illustrating an example of a stress state map showing a relationship among the vascular index value BV, the metabolic index value ME, and the stress state. An example of a determination method by the determination unit 104 will be described with reference to the stress state map illustrated in FIG. 4.

The determination unit 104 determines that the subject is in a stress state in a case where the relationship between the vascular index value BV and a threshold X1 satisfies a predetermined condition. For example, in a case where the vascular index value BV is a ratio of hexanoic acid to the total emission amount of hexanoic acid and propionic acid, when the vascular index value BV is equal to or less than the threshold X1, that is, when a point (BV, ME) is within a region R1 or a region R2 in a graph illustrated in FIG. 4, it is determined that the subject is in the stress state. The threshold X1 and the predetermined condition (such as a magnitude relationship between BV and X1) change in accordance with the selection of the vascular-related gas and the specific vascular-related gas.

In a case where it is determined that the subject is in a stress state, the determination unit 104 determines whether the subject is in a physical stress state where the subject feels physical stress or a mental stress state where the subject feels mental stress based on the relationship between the metabolic index value ME and the threshold Y1. For example, in a case where the metabolic index value ME is a ratio of the emission amount of acetone to the total emission amount of acetone, acetic acid, and acetaldehyde, when the vascular index value BV is equal to or less than the threshold X1 and the metabolic index value ME is equal to or greater than a threshold Y1, that is, when the point (BV, ME) is within the region R1 in the graph illustrated in FIG. 4, the subject is determined to be in the physical stress state.

Furthermore, the determination unit 104 determines that the subject is in the mental stress state in a case where the vascular index value BV is equal to or less than the threshold X1 and the metabolic index value ME is less than the threshold Y1, that is, in a case where the point (BV, ME) is within the region R2 in the graph illustrated in FIG. 4. The mental stress refers to a stress response caused psychologically by a stress stimulus (stressor), and refers to stress accompanied by symptoms such as a decrease in vitality, irritability, anxiety, and depression (for example, depressed mood, or decreased interest or concern).

Furthermore, the determination unit 104 determines that the subject is in a neutral state in a case where the vascular index value BV is greater than the threshold X1 and the metabolic index value ME is equal to or less than a threshold Y2, that is, in a case where the point (BV, ME) is within a region R3 in the graph illustrated in FIG. 4. The neutral state is not a stress state but may be a state where both the mind and the body are stable.

Furthermore, the determination unit 104 determines that the subject is in a temporary high activity state, in a case where the vascular index value BV is greater than the threshold X1 and the metabolic index value ME is greater than the threshold Y2, that is, in a case where the point (BV, ME) is within a region R4 in the graph illustrated in FIG. 4. The temporary high activity state is not a stress state corresponding to the region R1 and the region R2, but is a state where a physical load is higher than usual, and it can be regarded as a type of stress state. The conditions for setting the threshold values Y1 and Y2 and the regions R1 to R4 (such as the magnitude relationship among ME, Y1, and Y2) change in accordance with the selection of the metabolic-related gas and the specific metabolic-related gas.

According to the determination method as described above, it is possible to determine whether or not the subject is in the stress state based on the vascular index value BV. Furthermore, it is possible to determine whether the type of stress felt by the subject is a physical stress or a mental stress based on the metabolic index value ME in addition to the vascular index value BV. Furthermore, it is also possible to determine other states (for example, a neutral state) other than the stress states. Such a determination result regarding the other states can be used for generating, for example, stress information and support information.

In FIG. 4, a case where the stress state is classified into four states (regions), namely, a “physical stress state”, a “mental stress state”, a “neutral state”, and a “temporary high activity state”, is provided as an example. On the other hand, the number of stress states to be classified can be arbitrarily set as necessary.

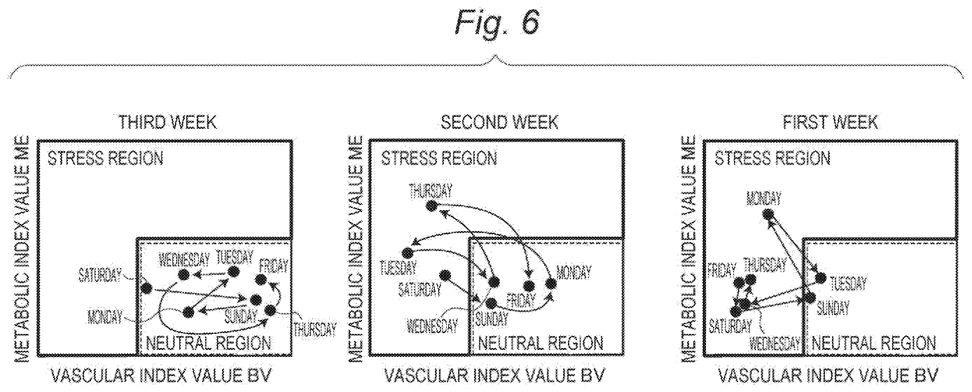

FIGS. 5 and 6 are diagrams illustrating other examples of a stress state map showing a relationship among the vascular index value BV, the metabolic index value ME, and the stress state. In the example illustrated in FIG. 5, the stress state is classified into two states, namely, a “stress state” and a “neutral (neutral) state”.

Furthermore, as indicated by black dots in the example of FIG. 6, it is also possible to identify a temporal change in the stress state by mapping the points (BV, ME) corresponding to the skin gases at different acquisition times onto the stress state map.

Furthermore, the determination unit 104 executes training suitability determination processing for a subject based on the determined stress state. Specifically, the determination unit 104 generates the training suitability classification information using the time-series information regarding the stress state, and executes the training suitability determination processing for the subject using the generated training suitability classification information. The training suitability classification information is information including a relationship between the training intensity and the temporal transition of the stress state.

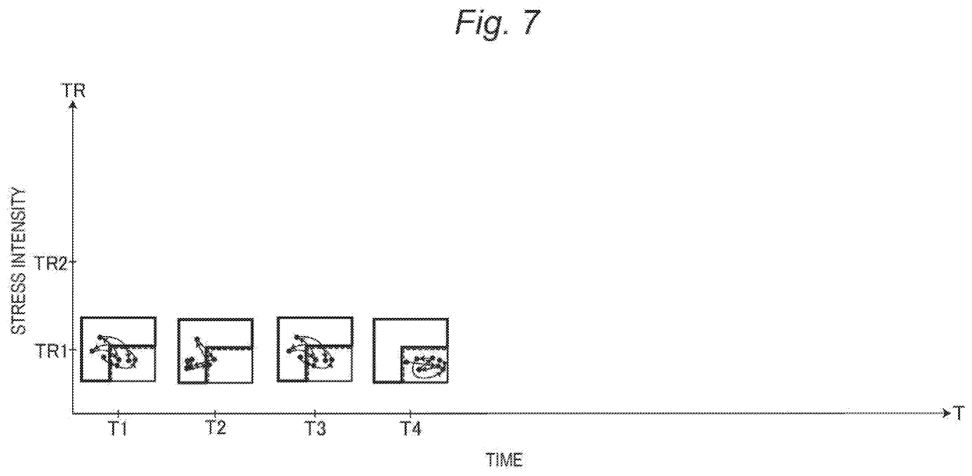

Here, the training suitability classification information and the training suitability determination processing using the training suitability classification information will be described in detail with reference to FIGS. 7, 8, and 9.

FIG. 7 is an example of a two-dimensional map showing a relationship between the training intensity of the subject and the time-series transition of the stress state, and is a diagram illustrating an example of the training suitability classification information. In the example of the training suitability classification information illustrated in FIG. 7, the mental and physical state two-dimensional maps illustrated in FIGS. 5 and 6 are disposed along a time series in a two-dimensional coordinate system in which the vertical axis indicates a training intensity TR and the horizontal axis is a time T. Each mental and physical state two-dimensional map includes a transition of the stress state over a week. T1 to T4 each indicate a temporal transition in units of one week. That is, each of the mental and physical state two-dimensional maps in the period from T1 to T4 corresponds to the transition of the stress state for four weeks in a case where the training is executed with a training intensity TR1.

As can be seen from the training suitability classification information illustrated in FIG. 7, in the periods of T1, T2, and T3, the stress state of the subject moves back and forth between a stress region (a state with stress or high stress) and a neutral region (a state with no stress or low stress). On the other hand, in the period T4, the stress state of the subject is almost in a neutral region (a state with no stress or low stress). It can be determined to be a state in which the subject is accustomed to the training with the training intensity TR1 (that is, a state that has transitioned from a medium-stress state to a low-stress state). In such a case, from the viewpoint of allostasis resilience, it is preferable to transition to a training intensity TR2 with high load in order to achieve a higher training effect.

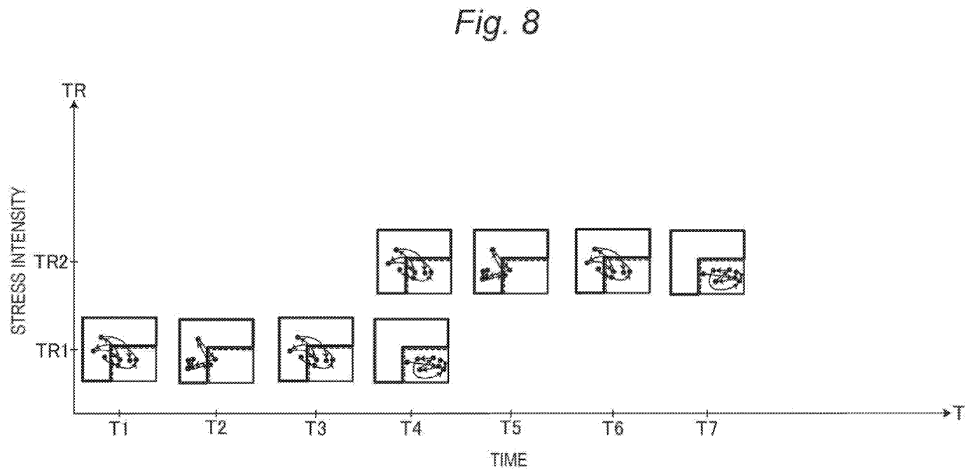

FIG. 8 is a diagram obtained by adding the transition of the stress state for four weeks in the case of executing the training over the period from T4 to T7 with the training intensity TR2 to the training suitability classification information illustrated in FIG. 7.

As can be seen from the training suitability classification information illustrated in FIG. 8, in the periods of T4, T5, and T6, the stress state of the subject moves back and forth between a stress region (a state with stress or high stress) and a neutral region (a state with no stress or low stress). On the other hand, in the period T7, the stress state of the subject is almost in a neutral region (a state with no stress or low stress). It can be determined to be a state in which the subject is accustomed to the training with the training intensity TR2 (that is, a state that has transitioned from a medium-stress state to a low-stress state). In such a case, from the viewpoint of allostasis resilience, it is preferable to transition to a training intensity TR3 with high load in order to achieve a higher training effect.

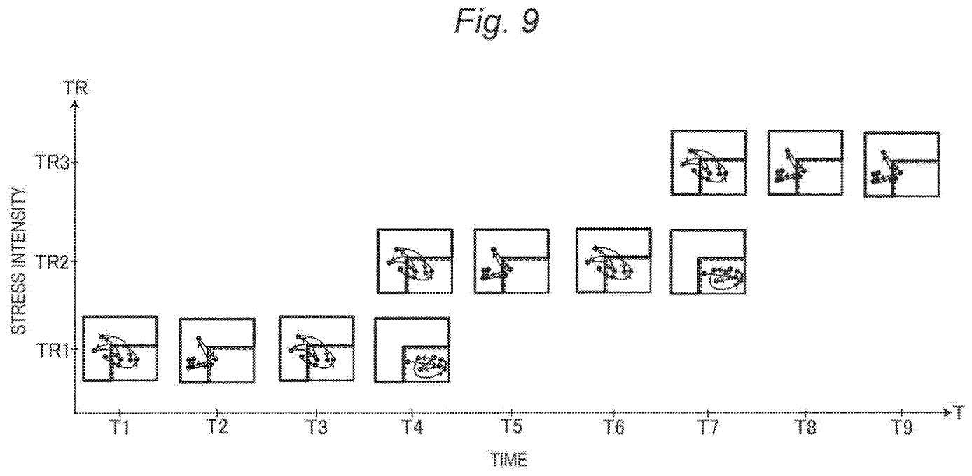

FIG. 9 is a diagram obtained by adding the transition of the stress state for three weeks in the case of executing the training over the period from T7 to T9 with the training intensity TR3 to the training suitability classification information illustrated in FIG. 8.

As can be seen from the training suitability classification information illustrated in FIG. 9, in the period of T7 to T9, the stress state of the subject moves back and forth between a stress region (a state with stress or high stress) and a neutral region (a state with no stress or low stress). Furthermore, in the periods of T8 and T9, the number of times the stress state of the subject is in the neutral region (a state with no stress or low stress) is not increased. It can be determined that the subject is in an overwork state (that is, the high stress state) as a result of continuing the training with the training intensity TR3. In such a case, from the viewpoint of allostasis resilience, it is preferable to take measures such as reducing the load or resting for a certain period of time in order to achieve a higher training effect.

The training suitability classification information is not limited to the examples illustrated in FIGS. 7 to 10. That is, the training suitability classification information may be any information as long as the training suitability can be determined from the temporal transition of the stress state.

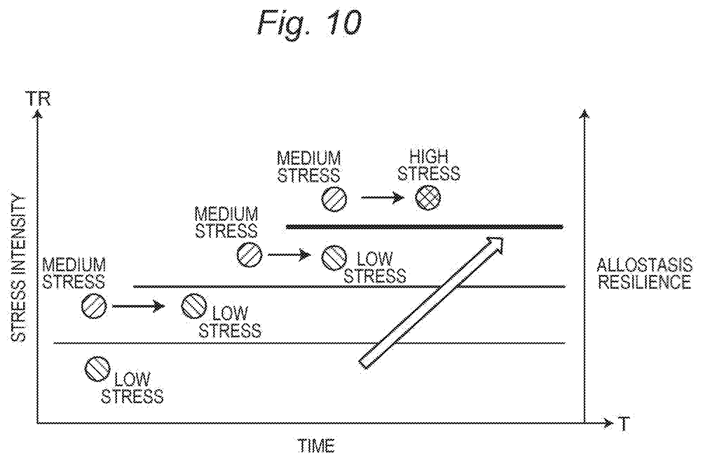

FIG. 10 is still another example of the two-dimensional map showing the relationship between the training intensity of the subject and the time-series transition of the stress state, and is a diagram illustrating still another example of the training suitability classification information. In the example of FIG. 10, each of the mental and physical state two-dimensional maps in FIGS. 7, 8, and 9 is abstracted into “low stress”, “medium stress”, and “high stress” from the transition of the stress state for a certain period of time in each of the mental and physical state two-dimensional maps, and mapped onto a two-dimensional coordinate system in which the vertical axis indicates the training intensity TR and the horizontal axis indicates the time T. The training suitability can also be determined from the temporal transition of the stress state based on the training suitability classification information illustrated in FIG. 10.

The determination unit 104 generates the training suitability classification information illustrated in FIGS. 7 to 10 by using a plurality of stress state maps including the latest stress state map and the training intensity corresponding to each stress state map. The determination unit 104 executes training suitability determination processing of determining whether or not the training currently performed by the subject is suitable using the generated training suitability classification information.

The generation unit 105 generates training support information regarding the subject based on the result of the training suitability determination processing by the determination unit 104.

Here, the training support information is information including at least one of a result of the training suitability determination processing for the subject or management information based on the result. For example, in a case where the determination unit 104 determines that the “training currently performed by the subject is not suitable”, the generation unit 105 generates, as the management information, information indicating that the currently performed training is unsuitable, information indicating an overtraining state, information indicating a high risk of injury or physical breakdown, and information indicating that rest for a predetermined period is required. Furthermore, for example, in a case where the determination unit 104 determines that “the training currently performed by the subject is suitable”, the generation unit 105 generates, as the management information, information indicating that the training currently performed is suitable, information such as “there is no risk of continuing the current training”, and the like.

The training support information may include information for reducing stress of the subject. Furthermore, the training support information can also include the training suitability classification information illustrated in FIGS. 7, 8, and 9.

Various methods can be adopted as a method for generating the training support information by the generation unit 105. For example, the generation unit 105 can generate the training support information by using a database (or a table) in which a plurality of results of a plurality of training suitability determination processing and a plurality of pieces of management information are associated with each other. Furthermore, the generation unit 105 can also generate the training support information using, for example, a trained model that receives the result of the training suitability determination processing and outputs the management information.

The output unit 106 outputs (presents) the training support information generated by the generation unit 105 in a predetermined format.

The storage unit 100 stores the skin gas information acquired by the acquisition unit 101, the vascular index value BV calculated by the vascular index value calculation unit 102, the metabolic index value ME, the determination result by the determination unit 104, the time-series information regarding the stress state determined in the past, the generated training suitability classification information, and the training support information for each subject.

The subject can simply and rapidly acquire and confirm the suitability of the current training and a specific measure for improvement using the presented training support information.

### Training Support Information Generation Processing

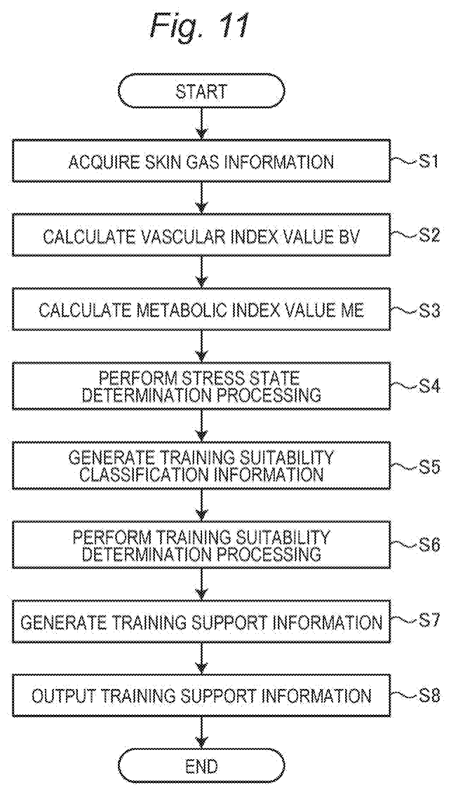

FIG. 11 is a flowchart illustrating an example of a flow of the training support information generation processing according to the embodiment. The training support information generation processing will be described with reference to FIG. 11.

First, the acquisition unit 101 acquires skin gas information from the skin gas sensor 11 (step S1).

The vascular index value calculation unit 102 calculates the vascular index value BV based on the skin gas information acquired by the acquisition unit 101 (step S2). The vascular index value BV is, for example, a ratio of an emission amount of hexanoic acid to a total emission amount of hexanoic acid and propionic acid.

The metabolic index value calculation unit 103 calculates the metabolic index value ME based on the skin gas information acquired by the acquisition unit 101 (step S3). The metabolic index value ME is, for example, a ratio of an emission amount of acetone to a total emission amount of acetone, acetic acid, and acetaldehyde.

The determination unit 104 executes stress state determination processing for the subject using the calculated vascular index value BV, the calculated metabolic index value ME, and the stress state map (step S4).

The determination unit 104 generates the training suitability classification information using the determined stress state and the time-series information related to the past stress state stored in the storage 24 (step S5).

The determination unit 104 executes the training suitability determination processing for the subject by using the generated training suitability classification information (step S6).

The generation unit 105 generates the training support information regarding the subject based on the result of the training suitability determination processing by the determination unit 104 (step S7).

The output unit 106 outputs the generated stress information in a predetermined format (step S8).

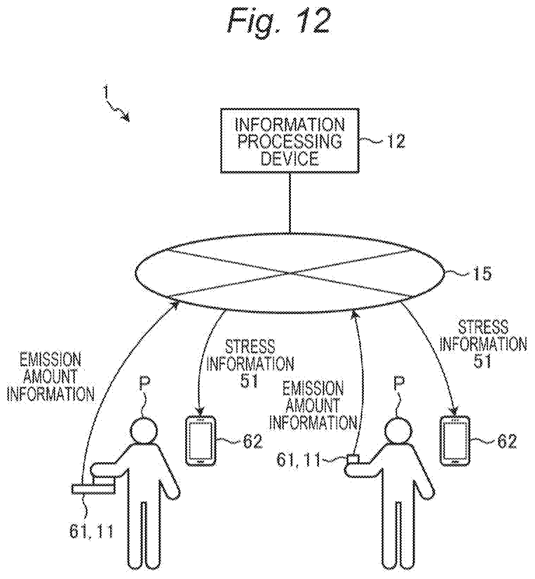

FIG. 12 is a diagram illustrating an example of a form of providing the training support information to the subject. In the training support information providing system 1 illustrated in FIG. 12, the training support information providing device 12 acquires the emission amount information corresponding to each of a plurality of subjects P from a predetermined detection device 61 via the network 15, generates training support information 51 of each subject P based on the acquired emission amount information, and transmits the generated training support information 51 to a terminal device 62 used by the corresponding subject P via the network 15.

The form of the detection device 61 (the device on which the skin gas sensor 11 is mounted) is not particularly limited, and may be, for example, a dedicated device fixedly installed at a specific place, a wearable device that can be worn by the subject P, or the like. The form of the terminal device 62 used by the subject P should not be particularly limited, and may be, for example, a smartphone, a tablet terminal, a personal computer, a dedicated device specialized for use in the training support information providing system 1, or the like.

As described above, the training support information providing device 12 according to the embodiment includes the acquisition unit 101 that acquires the emission amount information related to the emission amount of a plurality of types of skin gases emitted from the skin of the subject, the vascular index value calculation unit 102 and metabolic index value calculation unit 103 as calculation units that calculate at least one objective index value indicating a ratio of the emission amount of specific skin gas selected from the plurality of types of skin gases based on the emission amount information, the determination unit 104 that determines the stress state of the subject based on the at least one objective index value and determines the training suitability for the subject based on the determined stress state, the generation unit 105 that generates the training support information related to the subject based on the training suitability for the subject, and the output unit 106 that outputs the training support information.

Therefore, according to the training support information providing device 12, it is possible to correctly determine the stress state based on the objective index value calculated using the skin gas of the subject and to execute highly objective training suitability determination based on the determined stress state. Furthermore, it is possible to present specific and appropriate training support information for the subject based on highly objective training suitability determination. The subject can simply and rapidly acquire the training support information only by providing his/her skin gas, and can implement effective training using the training support information.

Furthermore, the training support information includes at least one of temporal transition of the stress state of the subject, training suitability classification information classified using the subjective index value of the subject and the objective index value of the subject, or management information based on the training suitability for the subject.

Therefore, according to the training support information providing device 12, it is possible to identify which specific stress suitability the subject belongs to. Furthermore, the subject can take specific measures to maximize the effect of the training using the management information based on the training suitability.

Furthermore, at least one index value includes at least one of a vascular index value or a metabolic index value.

Therefore, according to the training support information providing device 12, it is possible to determine the training suitability based on at least one of the physical stress state or the mental stress state, acquire the training support information based on the result thereof, and take specific measures.

### Second Embodiment

Next, a training support information providing device 12 according to a second embodiment will be described. The training support information providing device 12 according to the second embodiment generates training suitability classification information based on the subjective stress index value and the objective stress index value, and executes training suitability determination processing using the training suitability classification information. In the following description, a configuration different from that of the first embodiment will be described.

Here, the subjective stress index value is an index value quantified based on the stress felt by the subject. The subjective stress index value can be calculated by, for example, a method for causing the subject to answer a plurality of predetermined items and quantifying the plurality of predetermined items by a predetermined method.

Furthermore, the objective stress index value is an index value calculated based on chemical component analysis of skin gas. In the present embodiment, for specific description, a case where the objective stress index value is the vascular index value BV, the metabolic index value ME, or the like is provided as an example. However, the objective stress index value is not limited to the vascular index value BV and the metabolic index value ME, and may be other index values.

Furthermore, it is also possible to use a new index value calculated through arithmetic operations or the like of index values such as the vascular index value BV and the metabolic index value ME.

The storage unit 100 stores the subjective stress index value calculated in advance for each subject.

The determination unit 104 executes the training suitability determination processing for the subject based on the subjective stress index value and the objective stress index value. Specifically, the determination unit 104 generates the training suitability classification information for the subject by using the vascular index value BV calculated by the vascular index value calculation unit 102 or the metabolic index value E calculated by the metabolic index value calculation unit 103 and the subjective stress index value stored in the storage unit 100.

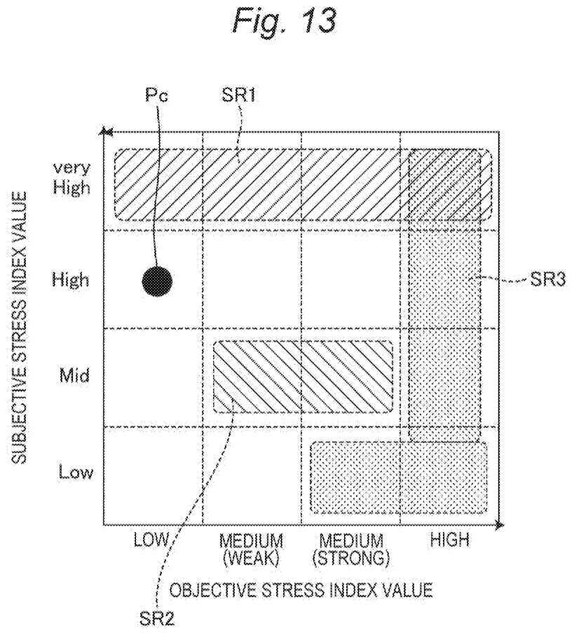

FIG. 13 is a diagram illustrating an example of the training suitability classification information according to the second embodiment. In the example of the training suitability classification information illustrated in FIG. 13, for example, a region R1 (a state with a high risk of overtraining syndrome), a region R2 (a state capable of representing high performance), and a region R3 (a state with a high risk of injury or physical breakdown) are set in the two-dimensional coordinate system in which the vertical axis indicates the subjective stress index value and the horizontal axis indicates the objective stress index value. Furthermore, a point Pc indicates the coordinates of (objective stress index value, subjective stress index value) of the subject.

Using the generated training suitability classification information, the determination unit 104 executes the training suitability determination processing of the subject from the positional relationship between the point Pc and each of the regions R1 to R3. For example, in a case where the point Pc is located in the region R1, the determination unit 104 determines that the current subject is in the “state with a high risk of overtraining syndrome”. Furthermore, in a case where the point Pc is located in the region R2, the determination unit 104 determines that the current subject is in the “state capable of representing high performance”. Furthermore, in a case where the point Pc is located in the region R3, the determination unit 104 determines that the current subject is in the “state with a high risk of injury or physical breakdown”.

The generation unit 105 generates the training support information regarding the subject based on the result of the training suitability determination processing by the determination unit 104.

For example, in a case where it is determined to be the “state with a high risk of overtraining syndrome”, the generation unit 105 generates training support information including management information indicating overtraining in order to move the position of the point Pc from the region R1 to the region R2.

Furthermore, for example, in a case where it is determined to be the “state capable of representing high performance”, the generation unit 105 generates training support information including management information such as “there is no risk of continuing the current training” in order to maintain the position of the point Pc in the region R2.

Furthermore, for example, in a case where it is determined to be the “state with a high risk of injury or physical breakdown”, the generation unit 105 generates the training support information including management information indicating a high risk of injury or physical breakdown in order to move the position of the point Pc from the region R3 to the region R2.

Furthermore, for example, as illustrated in FIG. 13, in a case where the position of the point Pc is not located in any of the regions R1 to R3, the training support information including the management information for moving the position of the point Pc from the region R3 to the region R2 is generated.

Furthermore, the training suitability classification information can be updated based on the temporal transition of the past stress state of the subject. For example, the region R1 (the state with a high risk of overtraining syndrome), the region R2 (the state capable of representing high performance), and the region R3 (the state with a high risk of injury or physical breakdown), which are illustrated in FIG. 13, vary along with the training result of the subject. The determination unit 104 can update the content of the training suitability classification information to be optimal for the current subject based on the temporal transition of the past stress state stored in the storage unit 107. The subject can receive the training support by using the training suitability classification information optimized in accordance with the variation in his/her training achievement.

The training support information generation processing executed by the training support information providing device 12 according to the second embodiment is substantially the same as that of the flowchart illustrated in FIG. 11.

According to the configuration described above, the training suitability classification information based on the subjective stress index value and the objective stress index value is generated, and the training suitability determination processing is executed using the training suitability classification information.

The training support information is generated based on the result of the training suitability determination processing and is provided to the subject.

Therefore, it is possible to simply and rapidly provide highly accurate training support information based on the subjective stress index value and the objective stress index value.

### Modification Example

In the above-described embodiment, the case where the determination of the stress state is executed using two indices of the vascular index value BV and the metabolic index value ME and the stress state map has been provided as an example. On the other hand, for example, it is also possible to determine the stress state using one of the vascular index value BV and the metabolic index value ME.

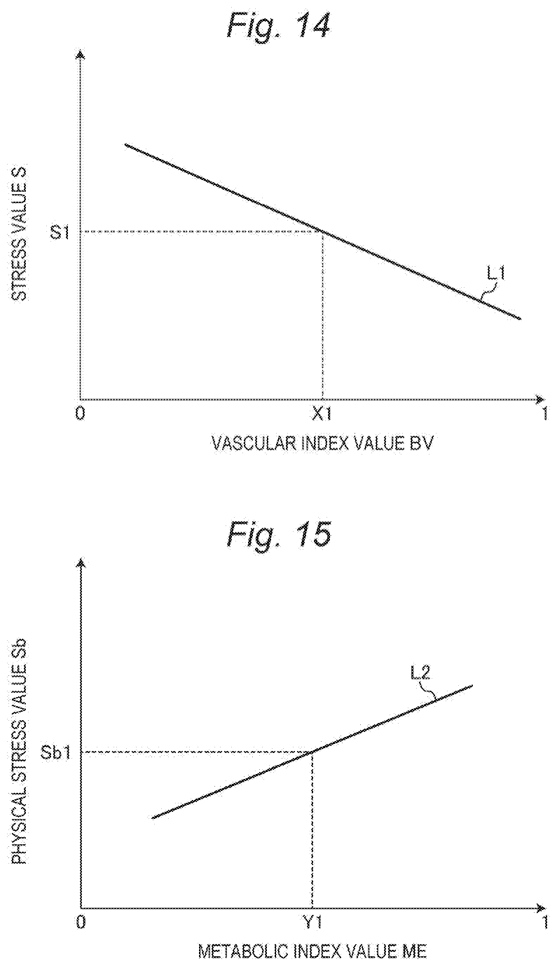

FIG. 14 is a diagram illustrating an example of a relationship between the vascular index value BV and a stress value S. In FIG. 14, a line L1 indicating the relationship between the vascular index value BV and the stress value S is illustrated. The stress value S is obtained by quantifying the intensity of the mental stress based on a predetermined criterion, and is set to be a higher value as the stress is stronger.

From FIG. 14, it can be seen that there exists a relationship in which the vascular index value BV becomes lower as the stress value S increases Here, in a case where the subject is set to be in a stress state when the stress value S is equal to or greater than a threshold S1, and in a case where the vascular index value BV is equal to or less than the threshold X1 corresponding to the threshold S1, it can be determined that the subject is in the stress state.

FIG. 15 is a diagram illustrating an example of a relationship between the metabolic index value ME and a physical stress value Sb. In FIG. 15, a line L2 indicating the relationship between the metabolic index value ME and the physical stress value Sb is illustrated. The physical stress value Sb is obtained by quantifying the intensity of the physical stress based on a predetermined criterion, and is set to be a higher value as the physical stress is stronger.

From FIG. 15, it can be seen that there exists a relationship in which the metabolic index value ME becomes higher as the physical stress value Sb increases Here, in a case where the subject is set to be in a physical stress state when the physical stress value Sb is equal to or greater than a threshold Sb1, and in a case where the metabolic index value ME is equal to or greater than the threshold Y1 corresponding to the threshold Sb1, it can be determined that the subject is in the physical stress state.

The determination unit 104 can also determine the stress state of the subject based on, for example, the relationship in FIG. 14 or 15, and execute mental and physical state classification processing of the subject based on the determined stress state.

According to the training support information providing device 12 according to the modification example, the determination of the stress state is executed using one of the vascular index value BV and the metabolic index value ME. Therefore, it is possible to reduce the calculation load and simplify the device as compared with the case of using both the vascular index value BV and the metabolic index value ME.

A program for causing a computer to execute processing for implementing the arithmetic function and the control function of the training support information providing device described above can be provided by being recorded on a computer-readable recording medium such as a compact disc (CD)-ROM, a flexible disk (FD), a CD-R (recordable), and a digital versatile disk (DVD) as a file in an installable format or an executable format. Furthermore, the program may be provided or distributed via a network such as the Internet.

Although the embodiments of the present disclosure have been described above, the above-described embodiments and modification example thereof are merely examples, and are not intended to limit the scope of the disclosure. The above-described novel embodiments and modification example can be implemented in various forms, and various omissions, substitutions, and changes can be made without departing from the gist of the disclosure. The above-described embodiments and modification example are included in the scope and gist of the disclosure, and are included in the disclosure described in the claims and the equivalent scope thereof.

### Summary of Present Embodiment

The present embodiment includes at least the following configuration.

The training support information providing device (12) according to the embodiment includes the acquisition unit (101) that acquires the emission amount information related to the emission amount of a plurality of types of skin gases emitted from the skin of the subject, the calculation units (102 and 103) that calculate at least one objective index value indicating a ratio of the emission amount of specific skin gas selected from the plurality of types of skin gases based on the emission amount information, the determination unit (104) that determines the stress state of the subject based on the at least one objective index value and determines the training suitability for the subject based on the determined stress state, the generation unit (105) that generates the training support information related to the subject based on the training suitability for the subject, and the output unit (106) that outputs the training support information.

Therefore, according to the training support information providing device (12), it is possible to correctly determine the stress state based on the objective index value calculated using the skin gas of the subject and to execute highly objective training suitability determination based on the determined stress state. Furthermore, it is possible to present specific and appropriate training support information for the subject based on highly objective training suitability determination. The subject can simply and rapidly acquire the training support information only by providing his/her skin gas, and can implement effective training using the training support information.

Furthermore, the training support information includes at least one of temporal transition of the stress state of the subject, training suitability classification information classified using the subjective index value of the subject and the objective index value of the subject, or management information based on the training suitability for the subject.

Therefore, according to the training support information providing device (12), it is possible to identify which specific stress suitability classification the subject belongs to. Furthermore, the subject can take specific measures to maximize the effect of the training using the management information based on the training suitability classification.

Furthermore, the training suitability classification information is updated based on the temporal transition of the past stress state of the subject.

Therefore, the subject can receive the training support by using the training suitability classification information optimized in accordance with the variation in his/her training achievement.

Furthermore, at least one index value includes at least one of a vascular index value or a metabolic index value.

Therefore, according to the training support information providing device 12, it is possible to determine the training suitability using at least one of the physical stress state or the mental stress state, acquire the training support information based on the result thereof, and take specific measures.

Although the embodiments of the present disclosure have been described above, the above-described embodiments and modification example thereof are merely examples, and are not intended to limit the scope of the disclosure. The above-described embodiments and modification example can be implemented in various other forms, and various omissions, substitutions, and combinations can be made without departing from the gist of the disclosure. Furthermore, the configuration and shape of each embodiment and each modification example can be partially interchanged.
(1) A training support information providing device includes an acquisition unit that acquires emission amount information related to an emission amount of a plurality of types of skin gases emitted from a skin of a subject, a calculation unit that calculates at least one objective index value indicating a ratio of an emission amount of specific skin gas selected from the plurality of types of skin gases based on the emission amount information, a determination unit that determines a stress state of the subject based on the at least one objective index value and determines training suitability for the subject based on the determined stress state, a generation unit that generates training support information related to the subject based on the training suitability determination for the subject, and an output unit that outputs the training support information.

According to the training support information providing device according to the embodiment, it is possible to effectively support the training of the subject such as an athlete by a simple and rapid method than in the related art.
(2) In the training support information providing device according to (1), the training support information includes at least one of temporal transition of the stress state of the subject, training suitability classification information classified using a subjective index value of the subject and the objective index value of the subject, or management information based on the training suitability for the subject.
(3) In the training support information providing device according to (2), the training suitability classification information is updated based on temporal transition of a past stress state of the subject.
(4) In the training support information providing device according to any one of (1) to (3), the at least one index value includes at least one of a vascular index value or a metabolic index value.

The principles, preferred embodiment and mode of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

1. A training support information providing device comprising:
an acquisition unit that acquires emission amount information related to an emission amount of a plurality of types of skin gases emitted from a skin of a subject;
a calculation unit that calculates at least one objective index value indicating a ratio of an emission amount of specific skin gas selected from the plurality of types of skin gases based on the emission amount information;
a determination unit that determines a stress state of the subject based on the at least one objective index value and determines training suitability for the subject based on the determined stress state;
a generation unit that generates training support information related to the subject based on the training suitability for the subject; and
an output unit that outputs the training support information.

2. The training support information providing device according to claim 1,
wherein the training support information includes at least one of temporal transition of the stress state of the subject, training suitability classification information classified using a subjective index value of the subject and the objective index value of the subject, or management information based on the training suitability for the subject.

3. The training support information providing device according to claim 2,
wherein the training suitability classification information is updated based on temporal transition of a past stress state of the subject.

4. The training support information providing device according to claim 1,
wherein the at least one index value includes at least one of a vascular index value or a metabolic index value.

5. The training support information providing device according to claim 2,
wherein the at least one index value includes at least one of a vascular index value or a metabolic index value.

6. The training support information providing device according to claim 3,
wherein the at least one index value includes at least one of a vascular index value or a metabolic index value.
